(19) United States
(12) Patent Application Publication
(10) Pub. No.: US 2021/0110903 A1
(43) Pub. Date: Apr. 15, 2021

(54) MEDICAL TRACKING SYSTEM

(71) Applicant: Jonathan Kost, Farmington, CT (US)

(72) Inventor: Jonathan Kost, Farmington, CT (US)

(21) Appl. No.: 16/532,475

(22) Filed: Aug. 5, 2019

(51) Int. Cl. G16H 20/10; G16H 70/40; G16H 40/67; G06F 16/2457; G06K 7/14

(52) U.S. Cl. CPC G16H 20/10; G16H 70/40; G06K 7/1417; G06F 16/2457; G16H 40/67

(57) ABSTRACT

The present tool creates a direct extension to patients outside and within the office setting. As used with a mobile device, the tool provides guidance on prescription of drugs or supplements as affected by a known genotype. The disclosed tool will collect, tabulate and report back to the medical practice or practitioner critical live-time information that will help in their medical decision-making. The collective data on patients may be segmented to evaluate various similar attributes among patients. The results of this collection of data may be graphically displayed to illustrate variants. Some examples may be the evaluation of diagnoses, guidance on prescription of drugs or supplements as affected by a known genotype, and the effects and side effects of a treatment on functionality, mental status and its associated complications and adverse reactions.

### CROSS-REFERENCE TO RELATED APPLICATION(S)

The present application claims priority to U.S. Provisional Patent Application No. 62/714,559 filed on Aug. 3, 2018, entitled “MEDICAL TRACKING SYSTEM” the entire disclosure of which is incorporated by reference herein.

### BACKGROUND

The present disclosure relates to the field of monitoring medical conditions and treatment, and, particularly, to analyzing data on medical treatment to determine risk factors and outcomes related to treatment.

In the past, electronic medical records have been largely maintained by a single doctor or medical establishment treating a patient, and (as generally being the property of the patients or subject to privacy laws) medical records rarely were shared. This led to complications for doctors treating new patients, as complicated questionnaires were developed to determine the medical condition and prior treatments of a new patient. If a mistake were made in the recollection of medical treatment, diagnosis of a condition could be less accurate or missed entirely. In addition, there was little consideration of previous drug treatment regimens.

Patients taking a multitude of drugs may suffer from drug interaction side effects, wherein a first drug causes an effect that renders a second drug useless or even harmful. While some of these negative drug interactions are known in advance and stored in tables, others are only determined by real-life anecdotal evidence of interaction, which may put patients in jeopardy. Evidence of adverse drug interactions may be best determined by drawing correlations from millions of interactions. However, privacy laws restricting access to patient medical records have, in the past, limited analysis of drug interactions.

Shopping for doctors often arises in connection with patients searching for a particular diagnosis or treatment from a first doctor. Those patients subsequently see a second doctor in order to receive a different, more preferable diagnosis or treatment. The second doctor will typically not know that the patient has already consulted the first doctor for the same issue(s) and may waste time and resources where a correct diagnosis was previously made by the first doctor. While each doctor may strive to provide the most accurate diagnosis to a patient, and seeing multiple doctors may reduce misdiagnosis from time to time, seeing multiple doctors may also be abused by certain patients, leading to inefficiencies in state and private medical insurance systems.

As a consequence of doctor shopping, some patients who receive drugs for pain may become addicted and may exhibit drug-seeking behavior. These patients may “shop” for doctors in order to obtain a new prescription for a pain medication they previously possessed. Alternatively, drug dealers may sell prescription medicines and use unscrupulous patients as the source for the drugs. There are some common databases that mark opiate prescriptions for tracking. However, due to privacy laws, limited information may be provided even when a match is found. In other cases, there are no systems for performing the matching, and drug-seeking patients are able to receive multiple prescriptions with impunity. In addition, there are many scenarios in which opioids may offer a beneficial treatment for pain, such as in post-surgery situations to treat patients with debilitating chronic pain. While opioid drugs face an ever-increasing stigmatization, especially in light of the recent opioid crisis, there is a population of patients that may benefit from effective treatment with opioids. Consequently, deciding whether to prescribe opioids may pose a quandary for the pain physician. In any case, outright elimination of opioid drugs is not the solution. Further, stigmatization of an entire therapeutic class (opioids) as being addictive for all patients is inaccurate and unwarranted. While it is important to ensure access to opioids for patients when it is medically warranted, providing access to opioids must be accomplished in a manner that prevents progression of the normal physiological dependence to pathological addictive disorders. Opioid prescription should be personalized, not forbidden. Personalization is the key to proper use of opioids, and it may provide insight into arresting the current opioid crisis.

Based on the foregoing, there is a need in the art for a system that is able to receive and record medical information for individual patients, including vital statistics, functional and psychological status and profile, doctor visits and diagnoses, drug regimens, including opioids and commonly abused drugs, as well as a history of interactions. Additionally, medical and surgical procedures would preferentially be recorded and correlated to produce unique data conclusions on treatment, best practices with associated side effects and complications involving post-treatment along with monitoring harmful drug combinations.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 illustrates a diagram showing the interface of pharmacokinetics and pharmacodynamics for opioid drugs.

FIG. 2 is a diagram which illustrates the molecular structure of selected opioid prodrugs and their effectiveness depending on various effectiveness characterizations of patient CYP2D6 metabolism.

FIG. 3 shows one embodiment of the invention illustrated in a flowchart according an exemplary process flow.

FIG. 4 shows one aspect of the invention as illustrated in a flowchart according to one exemplary process flow from the point of view of a patient/user

FIG. 5 illustrates a diagram showing high level communication interactions according to one aspect of an example of the foregoing.

FIG. 6 illustrates a block diagram of a communication system on which the foregoing may be implemented.

FIG. 7 illustrates a diagram representative of blockchain ledger system.

FIG. 8 illustrates a process flow according of a barcode scanner for reading supplement label information on supplement label.

### DETAILED DESCRIPTION OF EMBODIMENTS

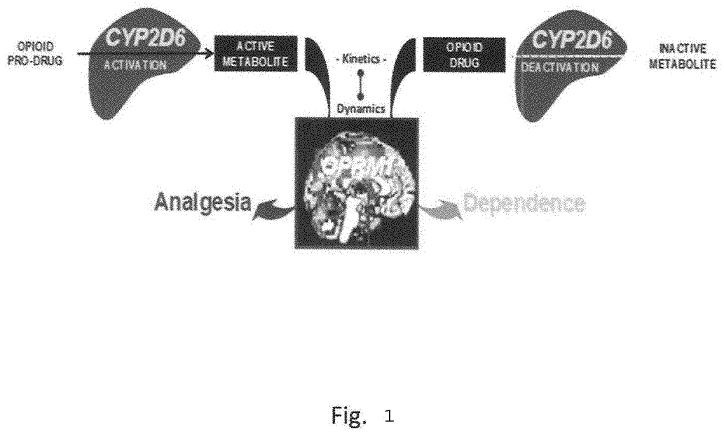

Embodiments of the present invention and their advantages may be understood by referring to FIGS. 1-8 wherein like reference numerals refer to like elements.

Variability in pharmacokinetics and pharmacodynamics is evidenced at the molecular level by genetic polymorphism. The medical adages describing pharmacokinetics as “what the body does to the drug”, and pharmacodynamics as “what the drug does to the body” remain useful concepts. Xenobiotic enzymes such as the cytochrome P450 (CYP450) family are the primary oxidative metabolizers of most drugs. Cytochrome P450 is encoded by the CYP2D6 gene in human beings. Drugs referred to as prodrugs, such as CYP2D6 Prodrugs, are activated by the action of CYP2D6. This enzyme also metabolizes several endogenous substances (such as hydroxytryptamines, neurosteroids, and both m-tyramine and p-tyramine which CYP2D6 metabolizes into dopamine in the brain and liver receptors), to endogenous ligands, such as the endorphins, which are the primary targets of opioids.

A recommended dose of a drug is designed to treat the “average” person. However, genetic variability has accumulated in humans since prehistoric times yielding enzymes and receptors that may offer results characterized by strikingly different blood levels, effectiveness and safety on an individualized basis. These variances are particularly relevant to opioid drugs used in pain control and they can now be measured by genotyping prior to treatment. Through clinical decision support interpreting genotyping data, drug choices and doses may be tailored to provide safe and effective drug therapy for individual patients. This precision affords personalized medicine to be practiced in pain treatment, especially pain treatment using opioid therapies.

CYP2D6 (cytochrome p450 2D6) and OPRM1 (μ1 opioid receptor) offer the most important genes coding, respectively, for a metabolizing enzyme and a receptor for opioids. The OPRM1 gene sends instructions for the production of a protein called the μ1 opioid receptor. Opioid receptors belong to the body's endogenous opioid system, which regulates pleasure, pain, and addictive tendencies.

Integration of genetic variability consideration in individual patient treatment provides a rational and scientific basis for personalized pain management. FIG. 1 illustrates a diagram showing the interface of pharmacokinetics and pharmacodynamics for opioid drugs. The cytochrome p450 2D6 isoenzyme, coded by the CYP2D6 gene and expressed primarily in the liver, is the enzyme primarily responsible for activation of opioid prodrugs (e.g. codeine, oxydocone) and deactivation of CYP2D6-substrate opioid drugs (e.g. meperidine). The μ1 opioid receptor, coded by the OPRM1 gene and expressed primarily in the brain, is the main target of both β-endorphins and opioid drugs (e.g. morphine, fentanyl, and methadone). The integration of molecular information, gained from genotyping, into heuristic and practical clinical analysis may be accomplished within a framework to support drug therapy considerations. Further, it may help provide examples that may help explain why genetic factors may make some patients more vulnerable than others to certain drug side effects.

It is possible to reliably assess the variation in the CYP2D6 gene. CYP2D6 is a hypervariable and hypermutable gene critically relevant to the pharmacogenetics of psychiatric and pain medications. CYP2D6's function is a major determinant of therapeutic response to opioids. CYP2D6 manifests duplications, rearrangements, deletions, and highly diverse haplotypes which characterize this gene. Besides the molecular complexity, there has been inconsistent assignment of function and nomenclature to the phenotypes (e.g., poor, intermediate, extensive, ultra-rapid) which has retarded widespread use of CYP2D6 genotyping. Many of the polymorphisms are bona fide mutations, rendering an important protein ineffectual. In one study, such CYP2D6 mutations are *3, *4, *4×N, *5 (deletion), *6, *7, *8, *11, *12, *14, *15 which account for a combined frequency of 20% in a referred population of 2406 primary care and psychiatric patients There are gene expansions that confer ultra-rapid CYP2D6 metabolizer status (*1×N, *2×N), accounting for 3% in the same cohort resolved at high resolution by expert haplotyping procedures.

Both null and expansion alleles, at 23% combined total frequency, are dysfunctional, as these bring metabolizer phenotypes to the extremes of function (poor and ultra-rapid, respectively). A simpler functional logic may be preferred, combining all the null mutations and gene expansions of CYP2D6 into a dysfunctional allele category which allows a heuristic application and direct guidance for the clinician. There are three practical functional CYP2D6 phenotypes in this logic: normal, subnormal and dysfunctional. The phenotypes, allele configurations and estimated prevalences based on the Hardy-Weinberg law are as follows:

CYP2D6 Functional (neither CYP2D6 allele is null or ultra-rapid) at 60% of the population,

CYP2D6 Subnormal (one CYP2D6 allele is null, the other normal) at 30%,

CYP2D6 Dysfunctional (both CYP2D6 alleles are null or at least one is ultra-rapid) at 10%.

In the context of prescribing opioids for pain management, determination of CYP2D6 gene variation is key to understanding inherent suitability for the patient, because of the effects of variants on drug metabolism. It has been shown that opioids have adverse events in patients at both extremes of function which may be characterized as ultra-rapid and poor. Further, in a survey of CYP2D6 metabolizer status at a specialized pain treatment center, it was found that these extremes tended to be enriched in the referred populations. It is for this reason, that both metabolizer extremes (ultra-rapid and poor) are considered dysfunctional and it is recommended that CYP2D6 substrate drugs and prodrugs be avoided in these patients.

Codeine, oxycodone, hydrocodone, and tramadol are opioid prodrugs with limited analgesic effect on their own as ingested. These prodrugs require hepatic CYP2D6 conversion to its most active metabolite to exert analgesia.

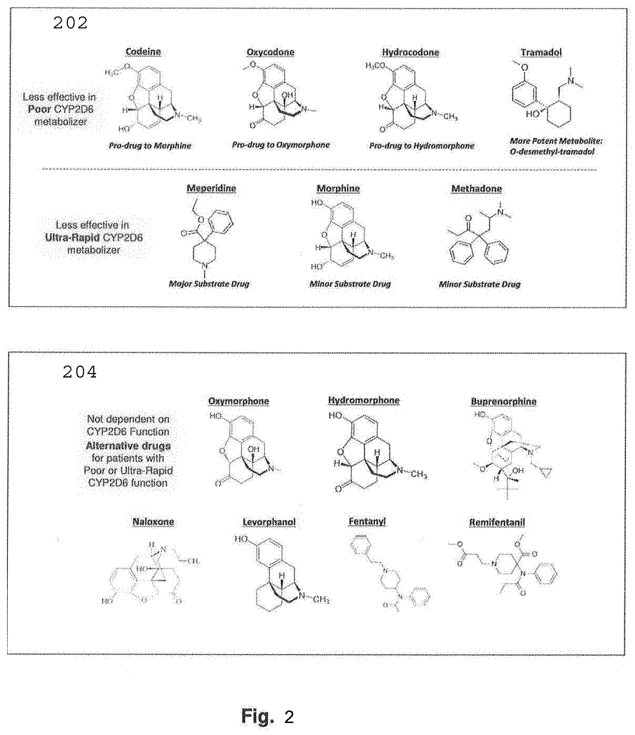

FIG. 2 is a diagram which illustrates the molecular structure of selected opioid prodrugs and their effectiveness depending on various effectiveness characterizations of patient CYP2D6 metabolism. In FIG. 2, panel 202 shows that codeine, oxycodone, hydrocodone, and tramadol are CYP2D6-substrate opioid prodrugs which require activation by CYP2D6 to their active metabolite (respectively, morphine, oxymorphone, hydromorphone and O-desmethyl-tramadol). For patients characterized as h poor CYP2D6 metabolizers, these prodrugs provide little, if any, analgesia to the patient. In FIG. 2, panel 202 also shows that meperidine (a major CYP-2D6 substrate opioid drug) is metabolized by CYP2D6 to a much lesser potent product. Additionally, morphine and methadone are minor CYP2D6 substrate opioid drugs which are partially metabolized by CYP2D6 to a less potent product. With patients characterized as ultra-rapid CYP2D6 metabolizers, meperidine, morphine and methadone provide decreased analgesia because of rapid deactivation. A person characterized as a CYP2D6 poor metabolizer obtains hardly any pain relief from the opioid prodrugs. For meperidine, which is an opioid drug primarily metabolized by CYP2D6 to an inactive metabolite, an ultra-rapid metabolizer would obtain less pain relief than a patient with normal function. Morphine and methadone, which are minor substrates for CYP2D6, are deactivated by CYP2D6 but only partially. Therefore, morphine and methadone are likely to be less effective with patients characterized as ultra-rapid metabolizers. Whether an opioid is a CYP2D6 prodrug or a drug which results in dysfunctional analgesic effects on a patient, it may provide extremes in assessing a patient's CYP2D6 functional status.

An ultra-rapid status presents unique risks for opioid treatments. For ultra-rapid metabolizers, prodrugs are metabolized in a burst as a bolus of active metabolite, producing serious side effects such as respiratory depression, and at best, an unsustainable response. The ultra-rapid status results in immediate breakdown, which prevents the attainment of therapeutic, steady-state drug concentrations.

Published cases of opioid toxicity due to codeine prescription in CYP2D6 ultra-rapid metabolizers are illustrative of this risk. In one non-lethal case, a cancer patient, with pneumonia, that was given codeine for cough suppression, went into respiratory arrest. Genotyping characterized the patient as a CYP2D6 ultra-rapid metabolizer with a functional gene expansion. Death was averted when the patient was treated with naloxone and fully recovered. In a tragic case, a newborn, of a mother taking codeine, died 13 days after birth. It was determined that breast milk from the mother, who was a CYP2D6 ultra-rapid metabolizer (with a functional gene duplication), was the inadvertent source of lethal doses of morphine for the baby. It has been suggested that codeine should be avoided in breast-feeding mothers who are ultra-rapid metabolizers of CYP2D6. The safety profile of codeine was reevaluated and the FDA issued a Black-box warning on codeine use in nursing mothers.

For the drugs metabolized to an inactive moiety, a rapid metabolizer obtains much less pain relief than a normal metabolizer. Rapid metabolizer patients may also evidence unusual psychiatric symptoms of opioid intoxication after codeine, hydrocodone, or oxycodone such as nervousness, restlessness, confusion, hallucinations, or paradoxical stimulation. In cases of dysfunctional CYP2D6, opioids or alternative analgesics which are not primary substrates of CYP2D6 should be considered. Opioids which are not CYP2D6 substrates include oxymorphone, hydromorphone, and buprenorphine. FIG. 2 shows, in panel 204, the chemical structural formula of non-CYP2D6 substrate opioid drugs. Drugs which are not substrates of CYP2D6 are not metabolized by CYP2D6 and are thus neither activated nor deactivated by the enzyme. Non-CYP2D6 dependent drugs should be considered preferentially in patients with dysfunctional CYP2D6 status. Examples of non-CYP2D6 substrate opioids include oxymorphone, hydromorphone, buprenorphine, naloxone, levorphanol, fentanyl, and remifentanil. These drugs still require monitoring for interactions with other drugs and diet, and the functional status of the patient for the opioid receptor OPRM1. These drugs may be preferred for individuals with dysfunctional CYP2D6 profiles.

It is becoming evident that pharmacodynamic genetic variants contribute to the development of opioid dependence. The p opioid receptor is the main target of both endogenous and clinically used opioids, such as oxycodone, their antagonists such as naltrexone, and an important mediator of drug dependence and opioid-induced respiratory depression. The μl-opioid receptor gene is the most widely studied gene in association with different aspects of chronic pain.

The μl opioid receptor gene (OPRM1) that codes for this receptor has a functionally significant and common variant termed A118G (rs1799971). This single nucleotide polymorphism (SNP) in exon 1 of the gene causes transition of an adenine (A) nucleotide to guanine (G) at base 118. In turn, the A118G transition at the DNA sequence causes the amino acid exchange at residue 40 of the p opioid receptor protein from the normal asparagine (Asn) to an abnormal aspartic acid (Asp) residue (Asn40Asp).

The Asp40 isoform of the receptor does not carry a N-glycosylation site in the extracellular region of the receptor, which reduces expression of the isoform at the cell surface, decreases μ-receptor binding potential in the brain, and increases morphine requirement (26). Significant reduction in effectivity of subsequent signaling pathways after the binding of specific agonists has been observed. The rate of G-protein coupling in carriers of the G allele is only half of that of AA homozygotes.

The global average frequency of the abnormal G allele is ˜20%, but with a wide population-specific range from 3% in individuals of African descent to nearly 50% in those of Asian descent. The phenotypes, allele configurations and estimated prevalences based on the Hardy-Weinberg law are as follows for a G allele frequency of 20%:

OPRM1 Functional (AA homozygous, both OPRM1 alleles are normal) at 64% of the population,

OPRM1 Subnormal (AG heterozygous, one OPRM1 allele is normal, one abnormal) at 32%,

OPRM1 Dysfunctional (GG homozygous, both OPRM1 alleles are abnormal) at 4%.

The respective isoform configurations at Asn40Asp are Asn/Asn (homozygous, functional), Asn/Asp (heterozygous, subnormal) and Asp/Asp (homozygous, dysfunctional).

Several studies evidence that having at least one copy of the G allele (AG or GG) is associated with lower pain threshold and higher opioid consumption in post-operative patients. This common protein coding polymorphism found in OPRM1 thus could render a patient less sensitive to opioid analgesic effects and more prone to dependence. In various clinical scenarios, patients with the G risk allele (Asp), rather than the normal A allele (Asn), appeared less sensitive to opioid medications. It is a trait that negatively could affect patient outcomes to opioid treatments.

A heuristic grounds and “rules of thumb” is preferable for integrating the foregoing based on basic principles of pharmacokinetics and pharmacodynamics. The rationale is based on observing the combinatorial configurations of CYP2D6 and OPRM1 in a given patient (Table 1) providing genotype matrix information indicating the advisability of prescribing certain drugs and opioid risk. Given three functional states for each CYP2D6 and OPRM1 gene, there are 9 combinatorial categories.

TABLE 1

| | | CYP2D6 | | | OPRM1 | FRE- |
|---|---|---|---|---|---|---|
| | | PRO- | CYP2D6 | | OPIOID | QUEN- |
| # | CYP2D6 | DRUGS | DRUGS | OPRM1 | RISK | CY |
| 1 | Functional | Prescribe | Prescribe | Functional | Low | 38.4% |
| 2 | Subnormal | High | Low | Functional | Low | 19.2% |
| | | Dose | Dose | | | |
| 3 | Dysfunc- | Non- | Non- | Functional | Low | 6.4% |
| | tional | CYP2D6 | CYP2D6 | | | |
| 4 | Functional | Prescribe | Prescribe | Subnormal | Moderate | 19.2% |
| 5 | Subnormal | High | Low | Subnormal | Moderate | 9.6% |
| | | Dose | Dose | | | |
| 6 | Dysfunc- | Non- | Non- | Subnormal | Moderate | 3.2% |
| | tional | CYP2D6 | CYP2D6 | | | |
| 7 | Functional | Prescribe | Prescribe | Dysfunctional | High | 2.4% |
| 8 | Subnormal | High | Low | Dysfunctional | High | 1.2% |
| | | Dose | Dose | | | |
| 9 | Dysfunc- | Non- | Non- | Dysfunctional | High | 0.4% |
| | tional | CYP2D6 | CYP2D6 | | | |

Table 1 shows a set of 9 combinatorial functional configurations CYP2D6 and OPRM1 based on a tri-fold functional status for each. The Functional status indicated for the gene (CYP2D6—neither allele is null or ultra-rapid; OPRM1—both alleles are normal, AA). The Subnormal status (CYP2D6—one allele is null, the other normal; OPRM1—one allele is abnormal, the other normal, AG). The Dysfunctional status (CYP2D6—both alleles are null or at least one is ultra-rapid; OPRM1—both alleles are abnormal, GG).

Guidance is provided in Table 1 for prescription, dosing, and selection of opioids, based on the combinatorial functional configuration. The estimated prevalence of each combinatorial configuration is based on the multiplication of individual frequencies for the functional categories of both genes (CYP2D6×OPRM1).

If a patient has functional CYP2D6 and OPRM1 phenotypes, the physician has carte blanche to implement judgment and experience garnered from previous patients.

When CYP2D6 is subnormal in a patient, dosing restrictions may be implemented determined by whether the medication is a drug or prodrug. If CYP2D6 is indicated as dysfunctional, non-CYP2D6 substrate drugs are recommended.

When a patient has OPRM1 subnormal or dysfunctional, the patient is less likely to benefit from opioids. There would be a higher incidence of side effects and dependence risk as well for these patients. Opioid doses should be carefully monitored in OPRM1 subnormal patients.

One would avoid escalating the opioid dose if a patient reports inadequate pain relief. Opioids should be avoided altogether in OPRM1 dysfunctional patients and reliance should be μlaced on non-opioids. Alternative electrophysiological and psychological therapies could be most useful in patients for whom drug therapy with opioids is ineffective or unsafe. Fortunately, modern pain management does afford also non-pharmacological treatments.

The foregoing approaches may be summarized into three clinical categories and priorities for genetically-guided opioid management, as follows:

### Clinical Category and Priority #1.

Patients at high-risk with dysfunctional CYP2D6 or OPRM1 account for ˜14% of the population [categories 3 (6.4%), 6 (3.2%), 7 (2.4%), 8 (1.2%), 9 (0.4%) in Table 1]. These patients must be identified early and may be treated with alternatives to opioids.

### Clinical Category and Priority #2.

Patients requiring opioid dose adjustment with subnormal CYP2D6 or OPRM1 account for ˜48% of the population [categories 2 (19.2%), 4 (19.2%), 5 (9.6%) in Table 1]. These patients are important to recognize for adjustments in opioid selection, dosing and monitoring of response.

### Clinical Category and Priority #3.

Patients likely to respond to standard opioid prescription and dosing with functional CYP2D6 and OPRM1 account for ˜38% of the population [category 1 (38.4%) in Table 1]. These are the patients who would suffer the most if disqualified from opioid therapy based on uninformed draconian prescription restrictions.

Personalizing pain management requires screening for opioid genetic alterations in the patient but also interfacing environmental triggers which interacts with the gene targets. Drug and diet interactions with metabolism inhibitors and inducers are the preeminent environmental modifier in the pharmacokinetic dimension. Co-prescription of any CYP2D6 strong inhibitors could generate an equivalent subnormal or dysfunctional phenotype and should be safeguarded with caution when opioids are being prescribed. Strong CYP2D6 inhibitors include antidepressants (e.g. bupropion, fluoxetine, paroxetine), antifungals (e.g. ketoconazole, miconazole) and antivirals (e.g. delavirdine, ritonavir). Dietary interactions are also significant, particularly with herbals. Some common dietary supplements interact with CYP450 and inhibit CYP2D6 (e.g. sesamin, turmeric). Lotus herbals (e.g. cosmetics, teas) significantly inhibit CYP2D6.

There are important clinical correlations that should inform the pharmacodynamic dimension for assessing opioid tolerance and dependence. Validated patient outcomes tools for pain management include PEG-3 (Pain, Enjoyment, General Activity) Scale, COMM-9 (Current Opioid Misuse Measure), and SOAPP (Screener and Opioid Assessment for Patients with Pain). As with all clinical symptoms, these represent the environmental modifiers. The genetic analysis offers perspective on the innate constitution of the individual as a baseline on which to superimpose environmental effects.

Toxicology urine drug screens are routine and valuable for monitoring the composite of environmental and clinical determinants of a patient's treatment. However, these are not sufficient for monitoring opioids to determine the patient's compliance or dependence. Even when performed at high resolution with mass spectrometry, urine toxicology will not be useful for phenotype-to-genotype correlations until the toxicology and genetic data are integrated into a clinical decision support system. Because of individual CYP2D6 metabolizer status, a fully compliant patient who is an ultra-rapid metabolizer could test negative for an opioid while a poor metabolizer could test consistently above the normal range.

In integrating pharmacokinetics and pharmacodynamics, the foregoing has been provided with consideration given to some very complex pathways of drug metabolism and activation while obviating others. The role of CYP3A4 in opioid metabolism is very significant for fentanyl, which is a major substrate, and secondary to CYP2D6 for codeine, hydrocodone, oxycodone and tramadol. CYP3A4 enzyme can be inhibited and the gene induced by multiple other drugs and diet. For example, macrolide antibiotics, azole antifungals, protease inhibitors and citrus juices are strong inhibitors, while rifamycins and anticonvulsants are strong inducers. CYP3A4 is far less genetically variable than CYP2D6, and is best managed by monitoring inhibitors and inducers that may be co-prescribed.

The foregoing concerns Phase I pharmacokinetics, and has not given consideration to Phase II and glucuronidation. In this domain, uridine diphospho-glucuronosyltransferase 2B7 (UGT2B7) is the predominant enzyme responsible for the glucuronidation of morphine to morphine-6-glucuronide (M6G) and morphine-3-glucuronide (M3G). The analgesic properties of morphine are enhanced by M6G and reduced by M3G. Polymorphisms in the gene encoding UGT2B7 may therefore have pharmacological, toxicological, and physiological significance.

As an example of the critical role of CYP2D6 genetic variation over CYP3A4 and UGT2B7 is the fact that the Clinical Pharmacogenetics Implementation Consortium guidance on opioids and CYP2D6 has a clear delineation of ultra-rapid and poor CYP2D6 function as a major risk for codeine prescription. The aforementioned guidance advises avoiding codeine altogether for patients with these extreme phenotypes, and suggests that to avoid treatment complications opioids that are not metabolized by CYP2D6, including morphine, oxymorphone, buprenorphine, fentanyl, methadone, and hydromorphone, along with nonopioid analgesics, may be considered as alternatives for use in CYP2D6 poor and ultrarapid metabolizers. The guidance further cautions that tramadol and, to a lesser extent, hydrocodone and oxycodone are not good alternatives because their metabolism is affected by CYP2D6 activity.

The classical distinction between pro-drugs and drugs rarely applies absolutely to opioids because both precursor molecule and metabolite are pharmacologically active to some degree. Nevertheless, CYP2D6 is the primary activator of codeine, oxycodone, hydrocodone, and tramadol whose metabolites are more potent analgesics than the parent molecule. Conversely, CYP3A4 is not involved in any conversion of parent opioid to a more potent metabolite. Hence for this initial exploration of genetic guidance for opioid prescription, CYP2D6 is the gene whose polymorphisms are of greatest clinical importance.

There is promising research indicating that other CYP450 genes (e.g. CYP3A4), opioid receptors (e.g., δ- and κ-receptors; genes OPRD1, OPRK1) and dopaminergic targets (e.g., dopamine receptor D2, transporter, and p-hydroxylase; genes DRD2, SLC6A3, DBH) may also contribute to a multi-gene model of response. However, functional variability in CYYP3A4 is primarily dependent on inhibition or induction by other drugs, rather than genetic polymorphism. Further, it has been demonstrated that among the opioid receptors, OPRM1 is the predominant predictor of opioid dependence. OPRM1 was involved in seventeen of eighteen models derived to predict opioid dependence (and in all the ten most statistically significant ones), while OPRD1 was involved in thirteen, and OPRK1 in six.

Including more genes in a model does not necessarily mean better prediction. As the roster of genes is increased, so do the possible environmental modifiers and disease comorbidities which may mask the innate gene effect of less predictive genes. Hence, multi-gene models invariably require very large populations to determine and validate the appropriate coefficients for each gene in a predictive matrix.

Some of these gene panels and interpretative algorithms are available in the market from commercial vendors. While genetic approaches may be represented in the commercial marketplace as a panacea, we believe physicians should also be informed about the inherent predictive limitations in models from vendors of multi-gene panels. Unfortunately, many of the algorithms in the market are maintained as trade secrets by the vendors, rendering it impossible for the physician to assess the predictive power or validity of the components in what effectively is a “black box”. Such overmarketing eventually backfires because uninformed utilization and increased costs lead to restrictions in coverage by insurance companies and to limitations in access for clinicians and patients.

Thus, even when such a multi-gene model has been obtained for a patient, it is recommended that the physician consider each gene result individually. The configuration of the main opioid pharmacokinetic and pharmacodynamic genes, CYP2D6 and OPRM1, should be assessed carefully because these remain the genes with the highest effect size and clinically predictive value based on the functional status as derived from their respective genotypes.

Personalization is important for all patients, but essential for those requiring preparation for starting or reversing a regimen of high-dose opioids or an unusual combination of agents. According to the National Center for Health Statistics, more than 63,600 lives were lost to drug overdose in 2016, the most lethal year yet of the drug overdose epidemic. Most of those deaths, 42,249 fatalities or 66%, involved opioids. Opioid abuse is the leading cause of mortality for people under age 50.

The current opioid crisis represents an alarming shift in the real outcomes of pain management from the patient's improvement in quality of life to iatrogenic drug dependence and addiction. Integral to the efficient and effective remediation of this crisis is the practice of personalized pain management, defined as the integrative assessment of clinical and genetic data for each individual. Legislative or legal moratoria on opioid use without prescription personalization can harm the public health at large by preventing physicians from treating the patients who can be predicted genetically to benefit the most from a proven and effective medication class.

Considerations of the cost-benefit of pharmacogenetic testing of pain patients should include the current reality that many primary care physicians are no longer treating pain patients and are opting to refer the patients to pain management specialty clinics. The evidence for cost effectiveness of pharmacogenetic testing relies on modeling potential savings versus the cost of genotyping. Currently, the current cost of genotyping hovers at ˜$500 per patient, which is high. However, genotyping can be effective when its cost is counterbalanced by savings in the health care system and services. Such studies examining clinic and prescription usage are under consideration. Drug waste from prescription changes and therapeutic management complications stemming from dysfunctional status could be avoided by pharmacogenetic guidance. The savings realized from genotyping could amortize the cost of testing, which itself is likely to come down. It is possible to device clinical criteria to select patients for genotyping and limit the testing initially to those with drug intolerance or ineffectiveness to reduce the cost of genotyping.

Necessary for this personalized pain medicine is a data storage, health informatics and reporting system that enable physicians to access patient outcomes and test results immediately following completion of treatments and laboratory processing. Such immediate and interactive presentation of outcomes and test results in an unambiguous, accessible and clinically actionable format will facilitate the translation of clinical data and laboratory results (including toxicology and genetics) to personalized pain management.

The present system may include an app or user interface that tracks a patient's status associated with a multitude of medical treatments, including medical and surgical procedures, along with medications, by using validated questions that assess functional state, pain level, psychological status, use of opioids, side effects to treatment medications and side effects to medical treatment. All of these results may be graphically displayed in real time and trended for the user. Further, these results may be sent back to a medical practitioner for simple interpretation, and addition to a patient medical file.

The data collected is used not only be used to assess patient progress, side effects of treatment and use (or overuse) of pain medications, it can be used assess all treatment outcomes of specific diagnoses, demographics, specific treatments, medications among other data elements that are desired to be collected. The data collected may include genotyping data for an individual user. This genotyping data may be securely stored on a mobile device or it may be stored remotely and accessed in connection with cloud storage. This may help preserve confidentiality and compliance with privacy laws. The app may be implemented in compliance with privacy laws. It may also possess a means for the patient to contact the practitioner site via email or chat for assistance or survey and treatment-related questions. The real-time results are then accessibly transmitted through a portal into the electronic medical records of the practitioner.

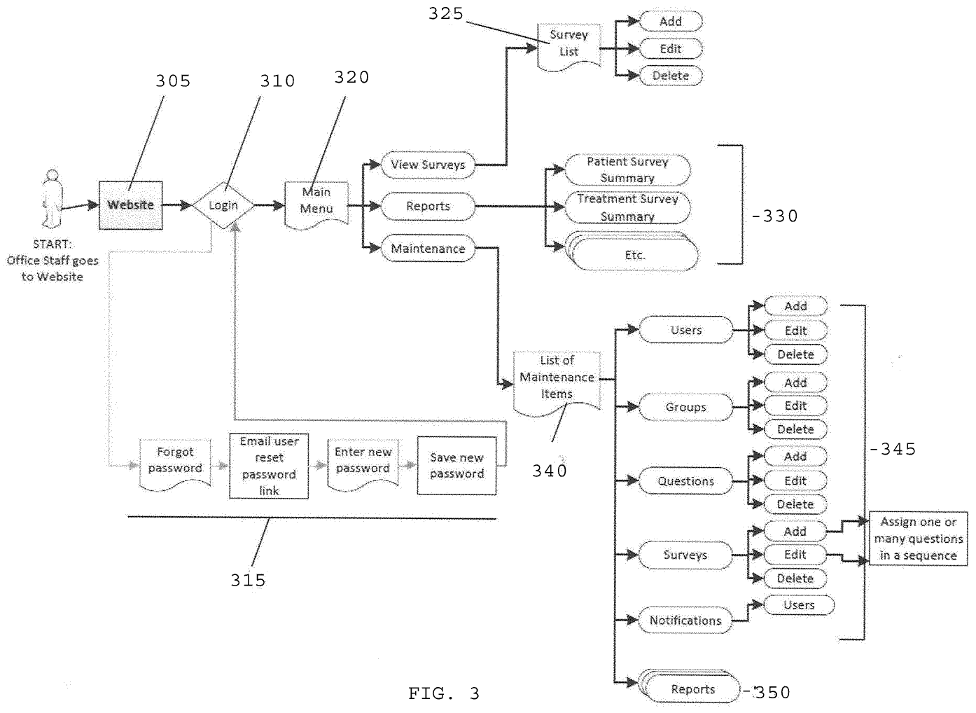

FIG. 3 shows one embodiment of disclosure herein illustrated in a flowchart according an exemplary process flow. With reference to FIG. 3, a software application (herein referenced as “app”) in step 305 is engaged by a user via a website visit. At step 310, the user logs in. If he/she has forgotten the password, there is a password reset procedure at step 315, wherein it is possible to reset a password in connection with an email link. At step 320, the user arrives at a main menu, in which he/she may select between several options, namely “view surveys”, “reports”, and “maintenance”. In connection with the “view surveys” option being chosen, the user is presented with a survey list at step 325. An option may be provided at this step which additionally permits additions, edits or deletions of a survey or survey questions. At this step, the interface may have some pre-selected survey questions that focus on eliciting some key health information from a patient. Responses to survey questions may be completed by the patient on his/her smartphone, smart-tablet, home computer, in-office smart-tablet, etc.

In connection with the “reports” option being chosen, at step 230 the user can select options such as patient survey summary, treatment survey summary, opioid medication summary and research diagnosis or treatment. If “patient survey summary” is chosen, a staff member receives a report of the patient survey results including treatment, prescriptions, subjective feelings, and other experiences associated with the treatment. These results are summarized, as well, to provide for economy of time for the staff. The individual and comparative treatment results may be overlaid against or compared to trends for risk factors, to identify i) complications from treatments or prescriptions, and ii) additional risk factors over time as a result of the treatment. Some risk factors associated with treatment are the side effects to medications which may have a high correlation to metabolic genetic anomalies, drug-to-drug interactions resulting in various side effects and complications, and post-procedural complications such as pain and infection. In some representative examples, the system may provide patients with educational documents and videos pertaining to a patient's diagnosis, medications, and treatments. In some examples, the system may provide a secure HIPAA-compliant email communication tool allowing communications between the patient and his/her medical provider. At the end of a medical treatment, a patient survey results may be generated, and patient survey results may be sent to a private company for monitoring quality of service and customer satisfaction.

In some examples, the system may include medical monitoring devices. One such device may include an accelerometer for use with a user's mobile phone. The accelerometer may measure walking running, bicycle riding distance and therefore provide some insight regarding the type and amount of physical activity or functionality that an individual is exemplifying over a period of time. This functionality could then be graphically displayed over time looking for trends and status after various medical treatments or interventions. A wearable device may provide walking distance, running distance, bicycle riding distance, sleep pattern and heart rate information as well as altitude information.

If the staff chooses “maintenance”, then at step 340 a list of maintenance items is provided for the staff to select from. At step 345 the staff may select “users”, “groups”, “questions”, “surveys”, “notifications”, each of which may be added to, edited or deleted. The “users” are either the patients or administrators (who possess variable levels of authority and access into the functioning of the system. At step 350 a reporting feature is available. This includes a report that graphically illustrates the patients survey results over time, mathematically calculating any validated test results, a further report that graphically illustrates selectable data related to multiple patients for research purposes and a third report that graphically illustrates opioid/medication utilization over time and will convert all opioid dosages into a common comparable dosage strength (Morphine Milliequivalent's).

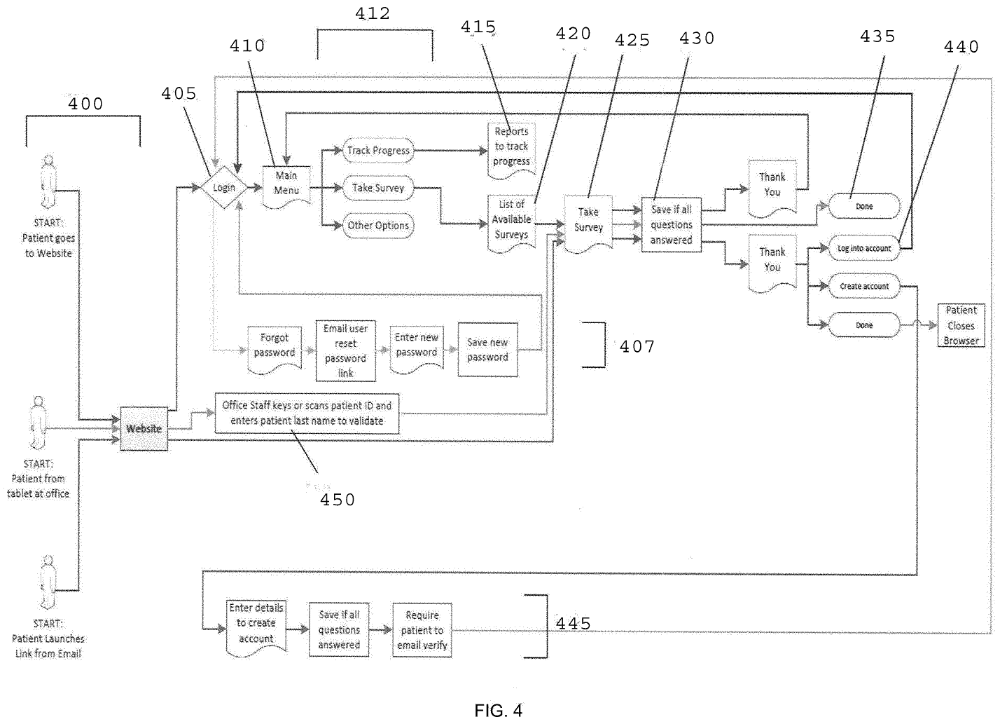

FIG. 4 shows one aspect of the invention as illustrated in a flowchart according to one exemplary process flow from the point of view of a patient/user. At step 400, the patient/user may visit a website; starts an app or website from a tablet; or launch a link to the website or app from an email message. Each of the foregoing involves the patient engaging the website for interaction. At step 405, the patient/user logs in. At step 407, if the patient/user has forgotten his/her password, the patient/user may be emailed a reset password link, which enables the patient/user, who has access to the email, to reset the password and save the new password. At step 410, the patient/user enters a main menu and at step 412 options for selection are displayed. such as “view progress”, “take survey” or other options, for example. At step 415, the patient/user has selected “view progress” and optionally receives a progress report that is reviewed by the doctor. At step 420, the patient/user has selected “take survey” and is provided with a list of available surveys, with a suggested survey that is pre-selected. At step 325, the patient/user may take a survey, and at step 430 if all questions are answered, the survey may be saved. At step 435, the patient/user is done with the survey, however at step 340 the patient/user may log in to save the survey, and if the patient/user does not yet have a log in id, he/she may create one at step 445, which comprises the steps of entering details to create an account, saving questions already answered, and requiring the patient/user to verify.

Once the patient/user has connected to the website or app at step 400, office staff may key or scan the patient ID and may enter the patient's last name, or other information, to validate the patient/user at step 450. The office staff can search for patients a variety of different ways including email, patient ID, first name, last name, and any/all of the other attributes like weight, age, work status, etc.

The system and app may also be used by pharmaceutical and medical device corporations and medical facilities to collect data, of which is HIPPA compliant, for the purposes of medical studies for the evaluation of efficacy, side effects and treatment comparisons.

When the app, according to the foregoing is used with a mobile device platform such as an Android™ or to an iOS™ platform, it also has the capacity of being collaborated with an accelerometer to also measure functionality level live-time status post an individuals' treatment.

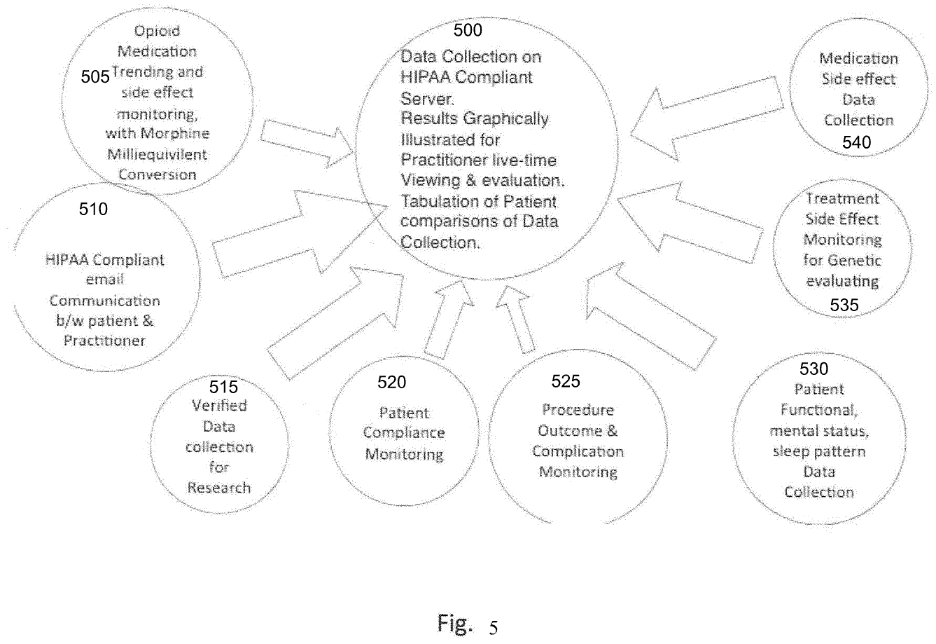

FIG. 5 illustrates a diagram showing high level communication interactions according to one aspect of an example of the foregoing. FIG. 5 shows modules providing information to the server 500. Opioid database 505 facilitates tracking of medication and side effects, and such information is provided to the server 500. Email system 510, which may be Health Insurance Portability and Accountability Act (HIPAA) compliant, provides email interfacing between doctor and patient, is a source of information to server 500. A database of verified data for research 515 communicates with server 500. A patient compliance monitoring system 520 also communicates with server 500. A procedure outcome and complication monitoring system 525 compiles data on complications and communicates it to the 500. A patient function and sleep pattern system 530 collects this data and communicates it to the server 500. A treatment side effect monitoring system 535 as well as a medication side effects system 540 provides data to server 500.

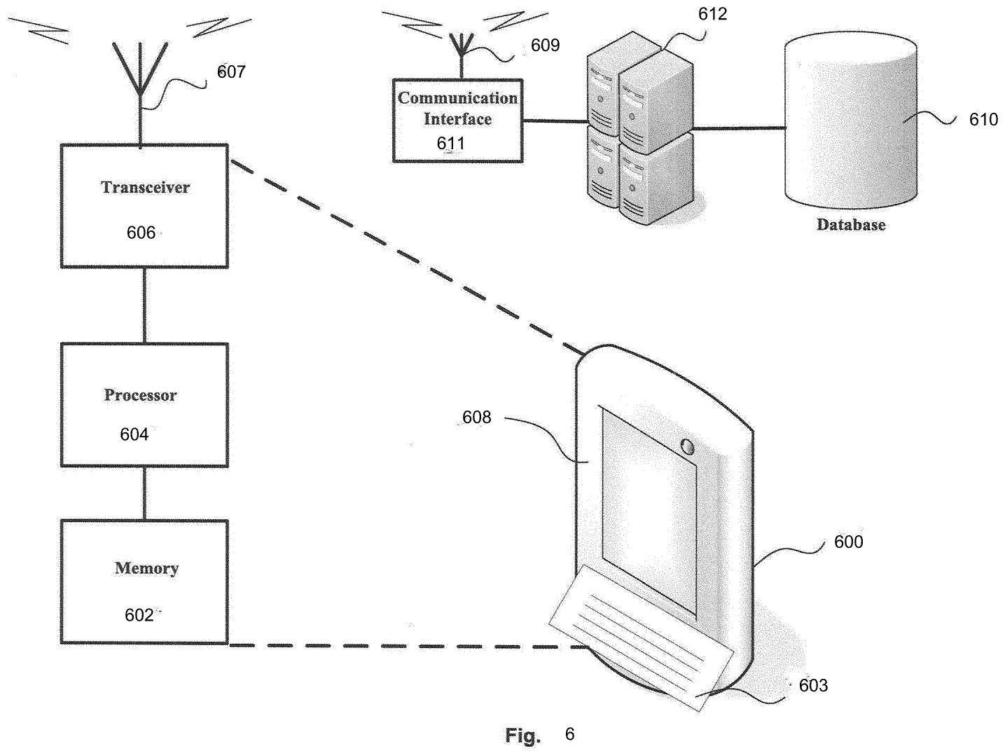

FIG. 6 illustrates a block diagram of a communication system on which the foregoing may be implemented. A user who has gone through the process of genotyping may store that genotyping data, under a protective password, within memory 602 on mobile device 600. In connection with reporting habits, food consumption, drug consumption, etc., through a user interface 603, processor 604 connected to memory 602 may dispatch genotyping data through transceiver 606 using antenna 607 to establish communication with a remote storage location such as server 612, through antenna 609 and communication interface 611, to access database 610 containing genotyping information such as that shown in Table 1 to provide guidance to avoid negative affects from usage of certain opioid medications. The guidance may be provided through server 612 through communications media such as the Internet or by a corresponding communication system to transceiver 606 (which may also be a separate transmitter and receiver). The results of the guidance may be passed through transceiver 606 to processor 604 to a user interface 603 and/or shown on display 608. Transceiver 606 is also contemplated as being a separate transmitter and a separate receiver. A User interfaces referenced herein may include audio information.

In another embodiment, the personal genotyping data may be stored in database 610 and accessed from mobile device 600, which may act as a thin client, using a password to server 612 which processes the stored personal genotyping data and genotyping information to produce guidance as determined by server 612, which may be relayed using the Internet or a communication system as described above, and assessed through a user interface 603 and/or shown on display 608.

In other embodiments, the genotype information of the type shown in Table 1, may be stored in database 610 which also includes stored personal genotyping data. Processor 604 may cause guidance on certain drug prescription or specific drug, food, or beverage intake in connection with processing the personal genotype data with the genotype information received in connection with accessing server 612. Results of the guidance may be communicated to user interface 603 or, display 608.

In yet another embodiment, the genotype information may be stored in memory 602 along with the personal genotype data. Processor 604 may cause guidance on certain drug prescription or specific drug, food, or beverage intake in connection with processing the personal genotype data with the genotype information. Results of the guidance may be communicated to user interface 603 or display 608.

In other embodiments, guidance may be obtained on mobile device 600 in connection with the mobile device accessing the genotype matrix information from a remote location at server 612 in connection with the guidance being arrived at using processor 604.

Processor 604 may run a program stored in memory 602 to carry out the functions described herein.

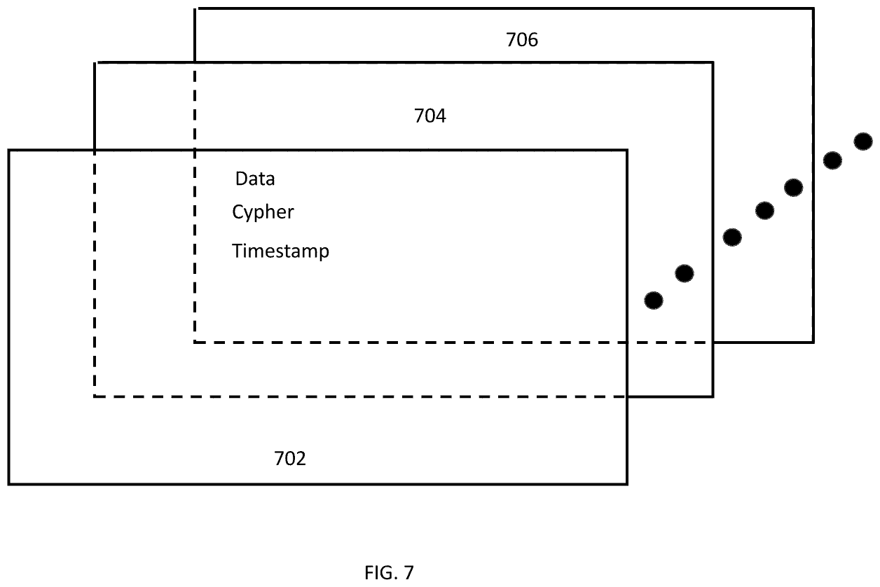

Rather than information being stored on centralized manner on database 610 it may alternatively be stored in a decentralized manner using a public or private blockchain ledger system. Such information storage occurs on a distributed basis. Preferably, the identity of a subject or patient is protected using a secure system which may include passwords of pseudonyms. FIG. 7 illustrates a diagram representative of blockchain ledger system 700. The first block, known as the genesis block 702 contains a hash of the data associated with the block. Thereafter, each block (704, 706, etc.) contains a time stamp, transaction data and a cryptographic hash of the previous block. The resulting block chain is resistant to modification through improper hashing as a cryptographic hash is a mathematical algorithm that may maps digital data of an arbitrary size to digital data of a fixed size. The cryptographic hash is often performed by what are known as miners that make use of graphic processors to properly perform the hash based off of the previous block. A ledger is embodied in the blockchain which may be shared among various entities in possession of the blockchain. In connection with a settlement period to update information of the block chain, the longest blockchain (and the blockchain with the most blocks) is retained and all shorter blockchains ae disposed of. The timestamp ensures that data is added to a block in the proper order.

In addition to concerns about opioids, nutritional supplements are of great concern as supplements are widely consumed in the USA without guidance as to need by the user. If properly used, these supplements can provide personalized healthcare and disease prevention for many patients with chronic conditions. A database is contemplated which contains the content of supplements by tradename and/or generic name. This database shall hereinafter be referenced as the supplements database and it may enable users who undergone genetic testing for cytochrome P450 enzymes. This database may be linked with a public online dietary supplement label database such as the Dietary Supplement Label Database (DSLD) provided by the National Institutes of Health (NIH). This database contains label information from over 7,600 dietary supplement product labels for identification of the supplements, including vitamins in the U.S. market.

Cytochrome P450 enzymes aid in the synthesis of steroid hormones, fats, and acids and may account for more than 70% of drug/supplement metabolizing enzymes. Cytochrome P450 enzymes may also metabolizes ingested medications and drugs/supplements. Approximately 60 cytochrome P450 genes may exist in a human body. In addition to polymorphisms negatively affecting the CYP2D6 gene, in connection with the use of opioids, polymorphisms may also negatively affect cytochrome P450 enzymes in connection with a patient/users use of drugs/supplements. The effects of polymorphisms may affect the breakdown of drugs/supplements. The rate of metabolism of the drug/supplement may depend upon a gene and its polymorphism (which may be identified in a patient through genetic testing). Drugs/supplements that are metabolize slowly stay active in a human body longer which may therefore require a lower dosage than otherwise needed. A faster metabolizing supplement may break down sooner and require a higher dosage for more beneficial effect.

Genetic testing of a patient relating to P450 enzyme polymorphism may be beneficially used to guide supplement use in connection with a tool (as carried out by a mobile device or computer application) that provides feedback in connection with processing data obtained through reading a barcode labeled on a supplement/drug container. This barcode, sometimes referred to as a QR™ code, may contain ingredient information for supplements and drugs obtain over-the counter and by prescription.

A barcode (QR scanner) and links to a dietary supplement label database may be built into a native platform on a mobile device. One specific embodiment links a patient's medications to indicate potential adverse drug interactions by inhibition of CYP450 enzymes by the dietary supplement.

In another embodiment, the tool as disclosed herein links the physical activity and functionality of the patient as a surrogate for disease status and/or disability. The activity is monitored via a wearable device. The wearable could be a remote sensor of patient activity (steps, walk, run, swim) such as FitBit™ or Apple™ Watch.

In yet another embodiment, links the drug regimen of the patient with potential inhibition of CYP2D6 by a vitamin or supplement may be provided by the tool disclosed herein. For example, a patient taking oxycodone for pain may experience less analgesia if ingesting Isoflavone-62, an anti-inflammatory supplement, or applying Lotus creams, a skin conditioner. Isoflavone and lotus are very strong inhibitors of CYP2D6, which enzyme is required to convert oxycodone to its active moiety oxymorphone. The tool may detect the presence of these substances in vitamin supplements or herbals, interface with the drug regimen, and alert the patient about precautions.

In still yet another embodiment, links the drug regimen of a patient with potential inhibition of CYP450 by a vitamin, supplement or herbal. For example, turmeric and sesamin are potent inhibitors of CYP2D6, CYP2C9, CYP2C19 and CYP3A4. Lotus is a very strong inhibitor of CYP2D6. Clinical repercussions of CYP450 inhibition are of utmost importance in pain management. A patient taking oxycodone for pain may experience less analgesia if ingesting turmeric, a spice with anti-inflammatory effects, or sesamin, a dietary fat-reduction supplement, or applying a Lotus cream, a skin conditioner. The loss of efficacy due to the CYP450 inhibition by these supplements stems from the fact that the CYP2D6 enzyme is required to convert oxycodone to its active moiety oxymorphone. The invention will identify the presence of these substances in vitamin supplements or herbals, interface with the drug regimen, and alert the patient about precautions.

Dietary recommendations received from the tool may be based on population and public health guidelines. The tool may allow those recommendations to be personalized and customized to the individual's medications and health status.

The native/app platform residing on a mobile device or accessed remotely online, may integrate medications, procedures, and treatments with laboratory results, genetic profiles, and patient-reported outcomes to produce guidance recommendations.

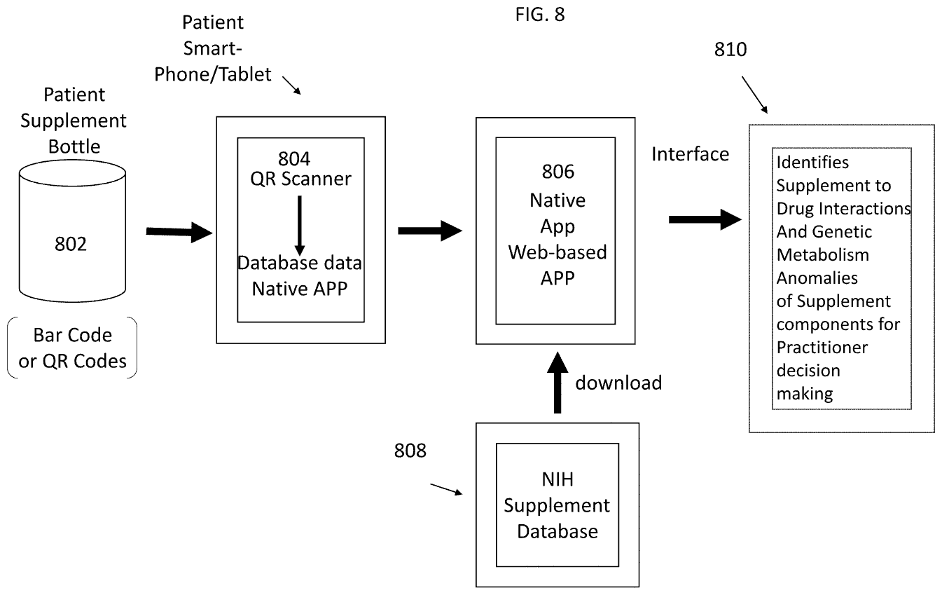

FIG. 8 illustrates a process flow according to the foregoing use of a barcode scanner for reading supplement label information on supplement label 802. At step 804, the label information from the barcode (QR) scan is processed by a processor on a mobile device running a native app. The label information may be stored in a database on the mobile device or transmitted to a processor located remotely from the device. At step 806 processor of the mobile device running the native app or remote processor causes a query of the DSLD database and receives relevant supplement information based on genetic testing information for a patient/user that may be securely stored in memory 602 (or accessed remotely through transceiver 606 of FIG. 6) and accessed by the processor (such as processor 604 in FIG. 6 or a processor located remotely and accessed by transceiver 606). At step 810 the relevant guidance and interactions may be communicated by, for instance transceiver 606 in FIG. 6, to an interface at for instance an emergency room device. This guidance may identify supplement to drug interactions and genetic metabolism anomalies of the supplement components to medical personnel such a decision making medical practitioner.

Hereinafter, general aspects of implementation of the systems and methods of the foregoing will be described. The system or portions of the system described herein may be in the form of a “processing machine,” such as a general-purpose computer, for example. As used herein, the term “processing machine” is to be understood to include at least one processor that uses at least one memory. The at least one memory stores a set of instructions. The instructions may be either permanently or temporarily stored in the memory or memories of the processing machine. The processor executes the instructions that are stored in the memory or memories in order to process data. The set of instructions may include various instructions that perform a particular task or tasks, such as those tasks described above. Such a set of instructions for performing a particular task may be characterized as a program, software program, or simply software.

As noted above, the processing machine executes the instructions that are stored in the memory or memories to process data. This processing of data may be in response to commands by a user or users of the processing machine, in response to previous processing, in response to a request by another processing machine and/or any other input, for example.

As noted above, the processing machine used to implement the foregoing may be a general purpose computer. However, the processing machine described above may also utilize any of a wide variety of other technologies including a special purpose computer, a computer system including, for example, a microcomputer, mini-computer or mainframe, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a Programmable Logic Device (“PLD”) such as a Field-Programmable Gate Array (“FPGA”), Programmable Logic Array (“PLA”), or Programmable Array Logic (“PAL”), or any other device or arrangement of devices that is capable of implementing the steps of the processes of the invention.

The processing machine used to implement the foregoing may utilize a suitable operating system. Thus, embodiments of the invention may include a processing machine running the iOS operating system, the OS X operating system, the Android operating system, the Microsoft Windows™ 8 operating system, Microsoft Windows™ 7 operating system, the Microsoft Windows™ Vista™ operating system, the Microsoft Windows™ XP™ operating system, the Microsoft Windows™ NT™ operating system, the Windows™ 2000 operating system, the Unix operating system, the Linux operating system, the Xenix operating system, the IBM AIX™ operating system, the Hewlett-Packard UX™ operating system, the Novell Netware™ operating system, the Sun Microsystems Solaris™ operating system, the OS/2™ operating system, the BeOS™ operating system, the Macintosh operating system, the Apache operating system, an OpenStep™ operating system or another operating system or platform.

It is appreciated that in order to practice the method of the foregoing as described above, it is not necessary that the processors and/or the memories of the processing machine be physically located in the same geographical μlace. That is, each of the processors and the memories used by the processing machine may be located in geographically distinct locations and connected so as to communicate in any suitable manner. Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing, as described above, is performed by various components and various memories. However, it is appreciated that the processing performed by two distinct components as described above may, in accordance with a further embodiment of the invention, be performed by a single component. Further, the processing performed by one distinct component as described above may be performed by two distinct components. In a similar manner, the memory storage performed by two distinct memory portions as described above may, in accordance with a further embodiment of the invention, be performed by a single memory portion. Further, the memory storage performed by one distinct memory portion as described above may be performed by two memory portions.

Further, various technologies may be used to provide communication between the various processors and/or memories, as well as to allow the processors and/or the memories of the invention to communicate with any other entity, i.e., so as to obtain further instructions or to access and use remote memory stores, for example. Such technologies used to provide such communication might include a network, the Internet, Intranet, Extranet, LAN, an Ethernet, wireless communication via cell tower or satellite, or any client server system that provides communication, for example. Such communications technologies may use any suitable protocol such as TCP/IP, UDP, or OSI, for example.

As described above, a set of instructions may be used with the processor(s) as described herein. The set of instructions may be in the form of a program or software. The software may be in the form of system software or application software, for example. The software might also be in the form of a collection of separate programs, a program module within a larger program, or a portion of a program module, for example. The software used might also include modular programming in the form of object-oriented programming. The software tells the processing machine what to do with the data being processed.

Further, it is appreciated that the instructions or set of instructions used in the implementation and operation of the invention may be in a suitable form such that the processing machine may read the instructions. For example, the instructions that form a program may be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processing machine, i.e., to a particular type of computer, for example. The computer understands the machine language.

Any suitable programming language may be used in accordance with the various embodiments of the invention. Illustratively, the programming language used may include assembly language, Ada, APL, Basic, C, C++, COBOL, dBase, Forth, Fortran, Java, Modula-2, Pascal, Prolog, REXX, Visual Basic, and/or JavaScript, for example. Further, it is not necessary that a single type of instruction or single programming language be utilized in conjunction with the operation of the system and method of the invention. Rather, any number of different programming languages may be utilized as is necessary and/or desirable.

Also, the instructions and/or data used in the practice of the invention may utilize any compression or encryption technique or algorithm, as may be desired. An encryption module might be used to encrypt data. Further, files or other data may be decrypted using a suitable decryption module, for example.

As described above, the invention may illustratively be embodied in the form of a processing machine, including a computer or computer system, for example, that includes at least one memory. It is to be appreciated that the set of instructions, i.e., the software for example, that enables the computer operating system to perform the operations described above may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in the invention may take on any of a variety of physical forms or transmissions, for example. Illustratively, the medium may be in the form of paper, paper transparencies, a compact disk, a DVD, an integrated circuit, a hard disk, a floppy disk, an optical disk, a magnetic tape, a RAM, a ROM, a PROM, an EPROM, a wire, a cable, a fiber, a communications channel, a satellite transmission, a memory card, a SIM card, or other remote transmission, as well as any other medium or source of data that may be read by the processors of the invention.

Further, the memory or memories used in the processing machine that implements the invention may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

In the system and method of the invention, a variety of “user interfaces” may be utilized to allow a user to interface with the processing machine or machines that are used to implement the invention. As used herein, a user interface includes any hardware, software, or combination of hardware and software used by the processing machine that allows a user to interact with the processing machine. A user interface may be in the form of a dialogue screen for example. A user interface may also include any of a mouse, touch screen, keyboard, keypad, voice reader, voice recognizer, dialogue screen, menu box, list, checkbox, toggle switch, a pushbutton or any other device that allows a user to receive information regarding the operation of the processing machine as it processes a set of instructions and/or provides the processing machine with information. Accordingly, the user interface is any device that provides communication between a user and a processing machine. The information provided by the user to the processing machine through the user interface may be in the form of a command, a selection of data, or some other input, for example.

As discussed above, a user interface is utilized by the processing machine that performs a set of instructions such that the processing machine processes data for a user. The user interface is typically used by the processing machine for interacting with a user either to convey information or receive information from the user. However, it should be appreciated that in accordance with some embodiments of the system and method of the invention, it is not necessary that a human user actually interact with a user interface used by the processing machine of the invention. Rather, it is also contemplated that the user interface of the invention might interact, i.e., convey and receive information, with another processing machine, rather than a human user. Accordingly, the other processing machine might be characterized as a user. Further, it is contemplated that a user interface utilized in the system and method of the invention may interact partially with another processing machine or processing machines, while also interacting partially with a human user.

The personalization of pain management is the key to curb the opioid crisis and genetics is a key component of that personalization. Genetics offers objective information on the innate baseline of the individual, upon which clinical characteristics and environmental modifiers could be integrated. Within the realm of utility, genetic predictions of opioid pharmacokinetics and pharmacodynamics are among the most accurate and clinically actionable because these reflect drug-gene interactions. The foregoing provides practical guidelines for implementation of CYP2D6 and OPRM1 polymorphisms concerning genetically-guided opioid prescription. This further provides risk-management stratification to safeguard patients least likely to benefit from opioids while identifying those who will likely benefit from opioid therapies. The foregoing allows a more informed choice for treatment of pain, providing a scientific justification for the selectivity used in prescribing opioid therapies rather than proscribing opioids altogether from a pain management regiment in all cases. The expected impact is a substantial reduction of dependence and increased efficacy of pharmacotherapy and procedures in the field of pain management.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention. The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A mobile device for providing guidance on prescription of drugs as affected by a known genotype comprising:
a memory for storing genotype data and genotype;
a transmitter for transmitting the genotype data to a remote location;
a receiver for receiving genotype guidance based on the stored genotype data; and
a display for conveying the guidance to a user.

2. A mobile device for providing guidance on prescription of drugs as affected by a known genotype comprising:
means for storing genotype data and patient genotype;
means for transmitting the genotype data to a remote location;
means for receiving genotype guidance based on the stored genotype data; and
means for conveying the guidance to a user.

3. The mobile device as recited in claim 2 wherein the means for storing genotype date including genotype includes distributed storage devices over one or more networks.

4. The mobile device as recited in claim 3 where the means for storing genotype data, including genotype, includes one or more blockchains systems.

5. A mobile device for providing guidance on the prescription of drugs affected by a known genotype, comprising:
means for storing genotype data and genotype matrix information;
means for accessing genotype information indicating the advisability of prescribing certain drugs and opioid risk;
means for processing the genotype data and the genotype matrix information to produce a guidance recommendation; and
means to convey the guidance recommendation to a user.

6. A non-transitory, computer-readable, programmable product, for use in conjunction with a processor, comprising code, executable by the processor, to cause the processor to do the following:
receive genotype data;
access a database containing genotype information indicating the advisability of prescribing certain drugs and opioid risk;
process the genotype data and the genotype matrix information to produce a guidance recommendation; and
convey the guidance recommendation to an electronic display.

7. A system for tracking and monitoring a patient's treatment, comprising:
means for storing genotype data and genotype matrix information;
means for accessing genotype information indicating the advisability of prescribing certain drugs and opioid risk;
means for processing the genotype data and the genotype matrix information to produce a guidance recommendation; and
means to convey the guidance recommendation to a user.

8. A non-transitory, computer-readable, programmable product, for use in conjunction with a processor, comprising code, executable by the processor, to cause the processor to do the following:
access genotype data from a memory;
receive barcode data from a barcode reader
produce a query for a remote database containing supplement guidance information based on the genotype data
receive guidance data from the remote database containing supplement guidance information; and
convey the guidance recommendation to an electronic display.

9. A system for providing supplement guidance comprising:
a mobile device for providing guidance for supplements as affected by a known genotype comprising:
a barcode reader for reading supplement label information
a memory for storing genotype data and genotype;
a transmitter for transmitting the genotype data to a remote location;
a processor for devising a query, based on the content of the supplement label information, to a database containing supplement information; and
a receiver for receiving supplement guidance information from the database containing supplement information.

10. A system for providing supplement guidance comprising:
a mobile device for providing guidance for supplements as affected by a known genotype comprising:
a barcode reader for reading supplement label information
a memory for storing genotype data and genotype;
a transmitter being operable to transmit the genotype data to a remote location;
a processor for devising a query, based on the content of the supplement label information, to a database containing supplement information; and
a receiver for receiving supplement guidance information from the database containing supplement information, the transmitter being further operable to convey the guidance information to a remote location.
